(19) United States
(12) Patent Application Publication
(10) Pub. No.: US 2023/0347169 A1
(43) Pub. Date: Nov. 2, 2023

(54) PHOTOTHERAPY DEVICE, PHOTOTHERAPY METHOD, AND COMPUTER-READABLE RECORDING MEDIUM

(71) Applicant: OLYMPUS CORPORATION, Tokyo (JP)

(72) Inventor: Chikashi OTA, Toyko (JP)

(73) Assignee: OLYMPUS CORPORATION, Tokyo (JP)

(21) Appl. No.: 18/220,362

(22) Filed: Jul. 11, 2023

Related U.S. Application Data
(63) Continuation of application No. PCT/JP2021/015612, filed on Apr. 15, 2021.

(51) Int. Cl. A61N 5/06; G06T 7/11

(52) U.S. Cl. CPC A61N 5/062; G06T 7/11; A61N 2005/0628; A61N 2005/0609; G06T 2207/10064; G06T 2207/30092; G06T 2207/20081; A61N 2005/0659

(57) ABSTRACT

A phototherapy device includes: a treatment light emitter configured to emit treatment light for causing a reaction of a drug; a first imager configured to obtain a tissue structure image which is formed using narrow band light applied onto an application position of the treatment light; a second imager configured to obtain a fluorescence image which is formed using excitation light applied onto the application position of the treatment light; a boundary region calculator configured to refer to the tissue structure image to determine a boundary region in which a tissue structure has changed; a fluorescence intensity variation calculator configured to calculate magnitude of variation in fluorescence intensity of the boundary region; and a display image generator configured to generate a display image to be used for displaying the magnitude of variation of the fluorescence intensity.

### CROSS-REFERENCE TO RELATED APPLICATION

This application is a continuation of International Application No. PCT/JP2021/015612, filed on Apr. 15, 2021, the entire contents of which are incorporated herein by reference.

### BACKGROUND

### 1. Technical Field

The present disclosure relates to a phototherapy device, a phototherapy method, and a computer-readable recording medium.

### 2. Related Art

In the related art, research is being carried out about photoimmunotherapy (PIT) in which an antibody drug is bound to cancer cells and is activated by the application of near-infrared light. As a result, the cancer cells get destroyed, and the cancer is treated (for example, refer to Japanese Patent Application Laid-open No. 2017-71654 and T. Nagaya, et al., Cancer Science. 2018; 109:1902-1908). The antibody drug, which has the near-infrared light applied thereto, absorbs the light energy; undergoes molecular oscillation; and produces heat. The probed cancer cells get destroyed due to that heat. At that time, the antibody drug produces fluorescence on account of becoming excited. The intensity of the fluorescence is used as an index of the effect of treatment.

### SUMMARY

In some embodiments, a phototherapy device includes: a treatment light emitter configured to emit treatment light for causing a reaction of a drug; a first imager configured to obtain a tissue structure image which is formed using narrow band light applied onto an application position of the treatment light; a second imager configured to obtain a fluorescence image which is formed using excitation light applied onto the application position of the treatment light; a boundary region calculator configured to refer to the tissue structure image to determine a boundary region in which a tissue structure has changed; a fluorescence intensity variation calculator configured to calculate magnitude of variation in fluorescence intensity of the boundary region; and a display image generator configured to generate a display image to be used for displaying the magnitude of variation of the fluorescence intensity.

In some embodiments, provided is a phototherapy method implemented for applying treatment light, which causes a reaction of a drug, onto a treatment area to confirm an effect of treatment, the phototherapy method includes: obtaining a tissue structure image which is formed using narrow band light applied onto an application position of treatment light; obtaining a fluorescence image which is formed using excitation light applied onto the application position of the treatment light; referring to the tissue structure image to determine a boundary region in which a tissue structure has changed; calculating magnitude of variation in fluorescence intensity of the boundary region; and generating a display image to be used for displaying the magnitude of variation of the fluorescence intensity.

In some embodiments, provided is a non-transitory computer-readable recording medium that stores a computer program to be executed by a phototherapy device applying treatment light, which causes a reaction of a drug, onto a treatment area to generate information to be used in confirming an effect of treatment. The program causes the phototherapy device to execute: obtaining a tissue structure image which is formed using narrow band light applied onto an application position of treatment light; obtaining a fluorescence image which is formed using excitation light applied onto the application position of the treatment light; referring to the tissue structure image to determine a boundary region in which a tissue structure has changed; calculating magnitude of variation in fluorescence intensity of the boundary region; and generating a display image to be used for displaying the magnitude of variation of the fluorescence intensity.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an overall configuration of an endoscope system according to a first embodiment of the disclosure;

FIG. 2 is a block diagram illustrating an overall configuration of the endoscope system according to the first embodiment of the disclosure;

FIG. 3 is a diagram for explaining a front-end configuration of an endoscope according to the first embodiment of the disclosure;

FIG. 4 is a diagram for explaining a configuration of an imaging optical system of the endoscope according to the first embodiment of the disclosure;

FIG. 5 is a diagram for explaining an example of the wavelength band of the light used as the narrow band light;

FIG. 6 is a diagram illustrating an exemplary flow of the treatment performed using the endoscope according to the first embodiment of the disclosure;

FIG. 7 is a flowchart for explaining an example of the operations performed by a processing device according to the first embodiment of the disclosure;

FIG. 8 is a diagram for explaining about the regions separated according to boundary region determination;

FIG. 9 is a diagram illustrating an exemplary transition of the fluorescence intensity when the reaction progress speed is slow;

FIG. 10 is a diagram illustrating an exemplary transition of the fluorescence intensity when the reaction progress speed is fast;

FIG. 11 is a block diagram illustrating an overall configuration of an endoscope system according to a modification example of the first embodiment of the disclosure;

FIG. 12 is a diagram for explaining a configuration of an imaging optical system of the endoscope according to the modification example of the first embodiment of the disclosure;

FIG. 13 is a diagram illustrating an overall configuration of an endoscope system according to a second embodiment of the disclosure;

FIG. 14 is a diagram for explaining a configuration of an imaging optical system of the endoscope according to the second embodiment of the disclosure;

FIG. 15 is a diagram that schematically illustrates an image obtained by a first imaging element;

FIG. 16 is a diagram that schematically illustrates an image obtained by a third imaging element;

FIG. 17 is a diagram for explaining the boundary region set as a result of combining the image illustrated in FIG. 15 and the image illustrated in FIG. 16;

FIG. 18 is a diagram illustrating an overall configuration of an endoscope system according to a third embodiment of the disclosure;

FIG. 19 is a diagram for explaining a configuration of an imaging optical system of the endoscope according to the third embodiment of the disclosure; and

FIG. 20 is a diagram illustrating an overall configuration of an endoscope system according to a fourth embodiment of the disclosure.

### DETAILED DESCRIPTION

Illustrative embodiments (hereinafter, embodiments) of the disclosure are described below with reference to the accompanying drawings. In the embodiments, as an example of a system that includes a phototherapy device according to the disclosure, the explanation is given about a medical endoscope system that takes images of the inside of a subject, such as a patient, and displays the images. Meanwhile, the disclosure is not limited by the embodiments described below. Moreover, in the drawings, identical constituent elements are referred to by the same reference numerals.

### First Embodiment

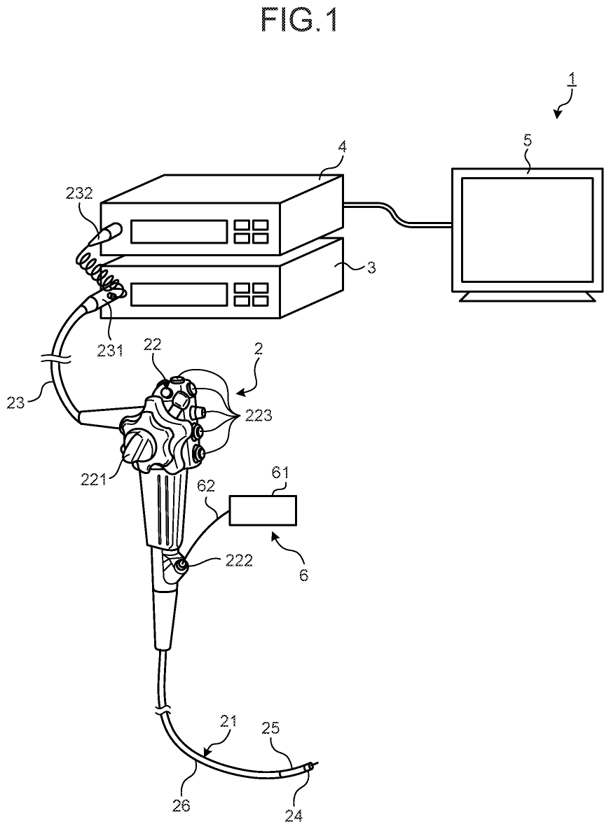

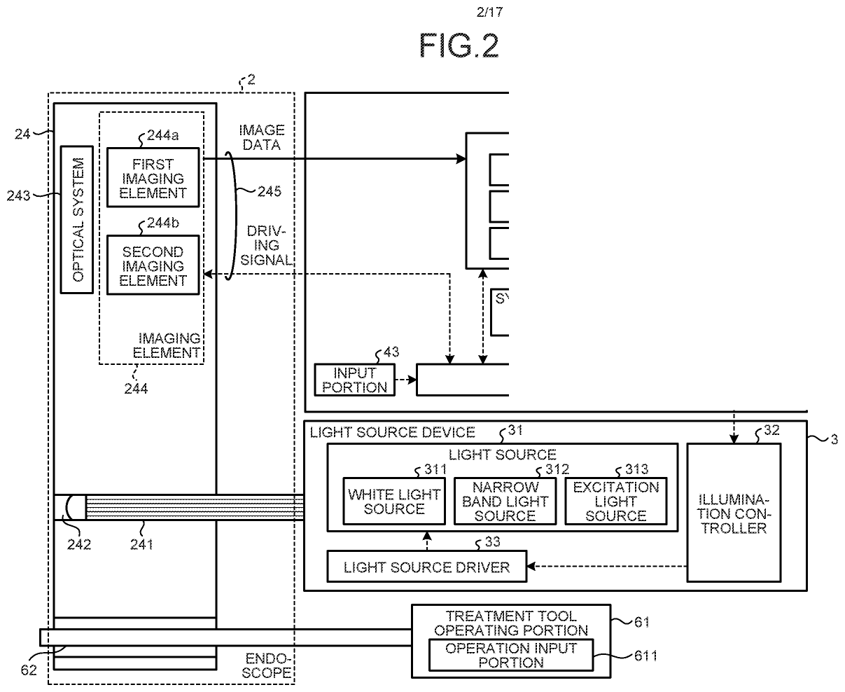

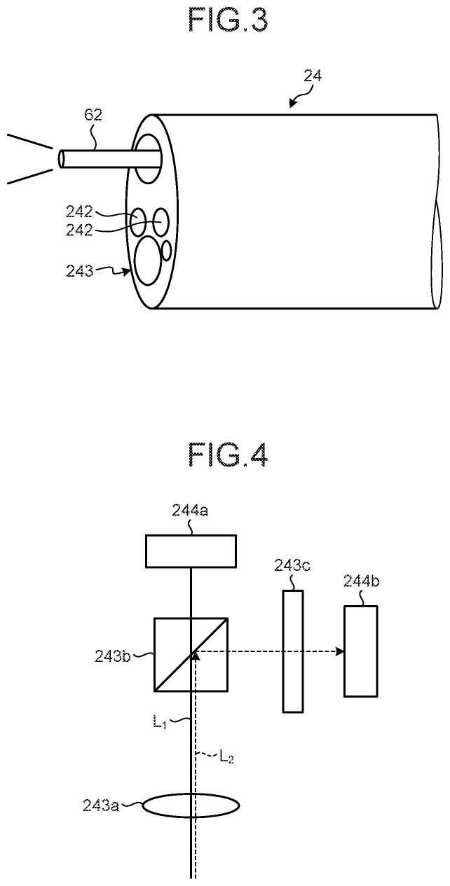

FIG. 1 is a diagram illustrating an overall configuration of an endoscope system according to a first embodiment of the disclosure. FIG. 2 is a block diagram illustrating an overall configuration of the endoscope system according to the first embodiment. FIG. 3 is a diagram for explaining a front-end configuration of an endoscope according to the first embodiment.

An endoscope system 1 illustrated in FIGS. 1 and 2 includes: an endoscope 2 that, when the front end portion thereof is inserted inside the subject, takes in-vivo images of the subject; a light source device 3 that generates an illumination light to be emitted from the front end of the endoscope 2; a processing device 4 that performs predetermined signal processing with respect to imaging signals that are obtained by the endoscope 2 by performing imaging, and that comprehensively controls the overall operations of the endoscope system 1; a display 5 that displays in-vivo images generated as a result of the signal processing performed by the processing device 4; and a treatment tool device 6.

The endoscope 2 includes: a flexible and elongated insertion portion 21; an operating portion 22 that is connected to the proximal end of the insertion portion 21 and that receives input of various operation signals; and a universal cord 23 that extends from the operating portion 22 in the opposite direction to the direction of extension of the insertion portion 21, and that has various built-in cables connected to the light source device 3 and the processing device 4.

The insertion portion 21 includes the following: a front end portion 24 that has a built-in imaging element 244 in which pixels meant for receiving light and generating signals according to photoelectric conversion are arranged in a two-dimensional manner; a freely-bendable curved portion 25 that is made of a plurality of bent pieces; and a flexible tube 26 that is a flexible and long tube connected to the proximal end of the curved portion 25. The insertion portion 21 is inserted into the body cavity of the subject. Then, using the imaging element 244, the insertion portion 21 takes images of the body tissue present at such positions inside the photographic subject up to which the outside light does not reach.

The operating portion 22 includes the following: a bending knob 221 that makes the curved portion 25 bend in the vertical direction and the horizontal direction; a treatment tool insertion portion 222 through which a treatment tool such as a treatment-light application device, biopsy forceps, an electrical scalpel, or an inspection probe is inserted into the body cavity of the subject; and a plurality of switches 223 representing operation input portions that receive input of operation instruction signals regarding the peripheral devices including not only the processing device 4 but also an insufflation unit, a water supply unit, and a screen display control. The treatment tool inserted from the treatment tool insertion portion 222 passes through a treatment tool channel (not illustrated) in the front end portion 24 and comes out from an opening of the front end portion 24 (see FIG. 3).

The universal cord 23 at least has a built-in light guide 241 and a built-in cable assembly 245, which has one or more cables bundled therein. The universal cord 23 is branched at the end portion on the opposite side to the side of connection with the operating portion 22. At the branched end portion of the universal cord 23, a connector 231 is disposed that is detachably attachable to the light source device 3, and a connector 232 is disposed that is detachably attachable to the processing device 4. From the end portion of the connector 231, some part of the light guide 241 extends out. The universal cord 23 propagates the illumination light, which is emitted from the light source device 3, to the front end portion 24 via the connector 231 (the light guide 241), the operating portion 22, and the flexible tube 26. Moreover, the universal cord 23 transmits the image signals, which are obtained as a result of the imaging performed by the imaging element 244 that is disposed in the front end portion 24, to the processing device 4 via the connector 232. The cable assembly 245 includes a signal line for transmitting imaging signals; a signal line for transmitting driving signals meant for driving the imaging element 244; and a signal line for sending and receiving information such as the specific information related to the endoscope 2 (the imaging element 244). In the first embodiment, the explanation is given under the premise that a signal line is used for transmitting electrical signals. Alternatively, a signal line can be used for transmitting optical signals, or can be used for transmitting signals between the endoscope 2 and the processing device 4 in a wireless manner.

The front end portion 24 is made of fiberglass, and includes the following: the light guide 241 that constitutes a light guiding path for the light generated by the light source device 3; an illumination lens 242 that is disposed at the front end of the light guide 241; an optical system 243 that collects light; and the imaging element 244 that is disposed at the image formation position of the optical system 243 and that receives the light collected by the optical system 243, performs photoelectric conversion, and performs predetermined signal processing with respect to electrical signals.

The optical system 243 is configured using one or more lenses. The optical system 243 forms an observation image on the light receiving surface of the imaging element 244. Meanwhile, the optical system 243 can also be equipped with the optical zooming function meant for varying the angle of view and the focusing function meant for varying the focal point.

The imaging element 244 performs photoelectric conversion with respect to the light coming from the optical system 243, and generates electrical signals (image signals). More particularly, the imaging element 244 includes two imaging elements (a first imaging element 244a and a second imaging element 244b). In the first imaging element 244a as well as the second imaging element 244b, a plurality of pixels, each of which includes a photodiode for storing the electrical charge according to the amount of light and includes a capacitor for converting the electrical charge transferred from the photodiode into a voltage level, are arranged as a two-dimensional matrix. In the first imaging element 244a and the second imaging element 244b, each pixel performs photoelectric conversion with respect to the incoming light coming via the optical system 243 and generates an electrical signal. Then, the first imaging element 244a as well as the second imaging element 244b sequentially reads the electrical signals generated by arbitrarily-set target pixels for reading from among a plurality of pixels, and outputs those electrical signals as image signals. The first imaging element 244a as well as the second imaging element 244b is configured using, for example, a CCD image sensor (CCD stands for Charge Coupled Device) or a CMOS image sensor (CMOS stands for Complementary Metal Oxide Semiconductor).

FIG. 4 is a diagram for explaining a configuration of an imaging optical system of the endoscope according to the first embodiment. The optical system 243 and the imaging element 244 are installed inside the front end portion 24.

The optical system 243 includes an objective lens 243a configured with one or more optical elements; a dichroic mirror 243b; and a cutoff filter 243c. The cutoff filter 243c cuts off the light having the wavelength band of the excitation light. Herein, the excitation light is equivalent to the light having the wavelength band meant for causing excitation of the antibody drug during photoimmunotherapy. Apart from including the optical elements mentioned above, the optical system 243 can also include lenses. Meanwhile, instead of using the dichroic mirror 243b, it is possible to use a beam splitter such as a half mirror.

The light coming from the photographic subject falls on the dichroic mirror 243b via the objective lens 243a. Herein, it is desirable that the distance from the light passing position/light returning position in the dichroic mirror 243b to the light receiving surface of each imaging element (the first imaging element 244a as well as the second imaging element 244b) is same.

The dichroic mirror 243b bends the light path of the light having the wavelength equal to or greater than the wavelength of the excitation light, and lets the light having the wavelength smaller than the wavelength of the excitation light pass through. At that is, the dichroic mirror 243b bends the light path of the excitation light meant for exciting the photographic subject and bends the light path of the fluorescence. The light that passes through the dichroic mirror 243b falls on the first imaging element 244a. On the other hand, the excitation light and the fluorescence having the light paths bent by the dichroic mirror 243b are cut off by the cutoff filter 243c, and the fluorescence falls on the second imaging element 244b.

The transmittance of the excitation light through the cutoff filter 243c is, for example, set to be equal to lower than 0.1%. As a result of setting the transmittance of the excitation light through the cutoff filter 243c to be equal to lower than 0.1%, the fluorescence can be selectively incorporated at the time of excitation light illumination.

The first imaging element 244a represents a first imager, and the cutoff filter 243c and the second imaging element 244b represent a second imager.

Meanwhile, the endoscope 2 includes a memory (not illustrated) that is used to store an execution program and a control program meant for enabling the imaging element 244 to perform various operations, and to store data containing the identification information of the endoscope 2. The identification information contains the specific information (ID), the model year, the specifications information, and the transmission method of the endoscope 2. Moreover, the memory can also be used to temporarily store the image data generated by the imaging element 244.

Given below is the explanation about a configuration of the light source device 3. The light source device 3 includes a light source 31, an illumination controller 32, and a light source driver 33. Under the control of the illumination controller 32, the light source 31 sequentially switches the illumination light and emits it onto the photographic subject (subject).

The light source 31 is configured using one or more light sources and one or more lenses, and emits a light (illumination light) when one of the light sources is driven. The light generated by the light source 31 is emitted from the front end of the front end portion 24 toward the photographic subject via the light guide 241. The light source 31 includes a white light source 311, a narrow band light source 312, and an excitation light source 313.

The white light source 311 emits the light having the wavelength band of the visible light range (i.e., emits a white light). The white light source 311 is implemented using an LED light source, a laser light source, a xenon lamp, or a halogen lamp.

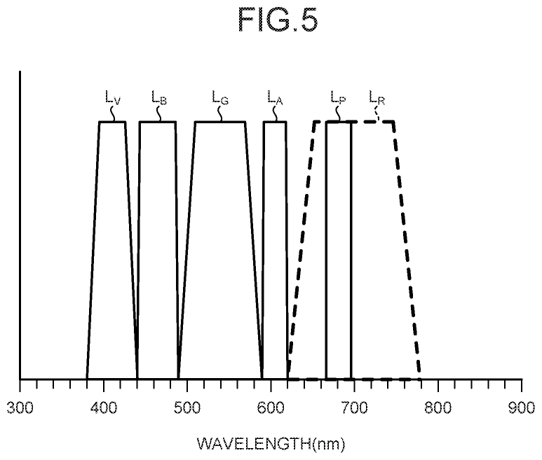

The narrow band light source 312 emits a light having some wavelengths or some part of the wavelength band from among the wavelength band of the visual light range. FIG. 5 is a diagram for explaining an example of the wavelength band of the light used as the narrow band light. The narrow band light is made of either one of the following lights or is made of a combination of some of the following lights: a light L_{V} having the wavelength band equal to or greater than 380 nm and equal to or smaller than 440 nm; a light L_{B} having the wavelength band equal to or greater than 440 nm and equal to or smaller than 490 nm; a light L_{G} having the wavelength band equal to or greater than 490 nm and equal to or smaller than 590 nm; a light L_{A} having the wavelength band equal to or greater than 590 nm and equal to or smaller than 620 nm; and a light L_{R} having the wavelength band equal to or greater than 620 nm and equal to or smaller than 780 nm. Examples of the narrow band light include the following lights used in NBI observation (NBI stands for Narrow Band Imaging): the light having the wavelength band equal to or greater than 380 nm and equal to or smaller than 440 nm, with the central wavelength of 415 nm; and the light having the wavelength band equal to or greater than 490 nm and equal to or smaller than 590 nm, with the central wavelength of 540 nm. The narrow band light source 312 is implemented using an LED light source or a laser light source.

Meanwhile, in the case of causing excitation of the antibody drug during photoimmunotherapy, for example, a near-infrared light Le having the central wavelength of 690 nm is used.

Herein, if the light having the wavelength equal to or greater than 380 nm and equal to or smaller than 440 nm is emitted and if the scattering light or the returning light is obtained, then the blood vessels in the superficial portion of the mucous membrane can be visualized with a high degree of contrast. Alternatively, if the light having the wavelength band equal to or greater than 490 nm and equal to or smaller than 590 nm is emitted, or if the light having the wavelength band equal to or greater than 590 nm and equal to or smaller than 620 nm is emitted, or if the light having the wavelength band equal to or greater than 620 nm and equal to or smaller than 780 nm is emitted, and if the scattering light or the returning light is obtained; then the blood vessels in the relatively deeper portion of the mucous membrane can be visualized with a high degree of contrast.

The light having the wavelength band equal to or greater than 440 nm and equal to or smaller than 490 nm is used not only for the visualization of blood vessels but also as a reference light in, for example, generating images meant for correcting the fluorescence intensity.

Meanwhile, in the case of using the light having the wavelength intensity equal to or greater than 620 nm and equal to or smaller than 780 nm; either the dichroic mirror 243b in the optical system 243 is replaced with a half mirror, or the optical system 243 is not modified but the electrical signals generated by the second imaging element 244b are used.

The excitation light source 313 emits the excitation light meant for causing excitation of the excitation target (for example, an antibody drug during photoimmunotherapy). The excitation light source 313 is implemented using an LED light source or a laser light source. In the case of causing excitation of an antibody drug during photoimmunotherapy, for example, the near-infrared light L_{P} is used.

Based on a control signal (modulated light signal) received from the processing device 4, the illumination controller 32 controls the electrical energy to be supplied to the light source 31, controls the light source to be made to emit light, and controls the driving timing of the light source.

Under the control of the illumination controller 32, the light source driver 33 supplies an electrical current to the light source to be made to emit light, and causes the light source 31 to output the light.

Given below is the explanation of a configuration of the processing device 4. The processing device 4 includes an image processor 41, a synchronization signal generator 42, an input portion 43, a controller 44, and a storage 45.

The image processor 41 receives, from the endoscope 2, image data of the illumination light of each color as obtained by the imaging element 244 by performing imaging. If analog image data is received from the endoscope 2, then the image processor 41 performs A/D conversion and generates digital imaging signals. Moreover, if image data in the form of optical signals is received from the endoscope 2, then the image processor performs photoelectric conversion and generates digital image data.

The image processor 41 performs predetermined image processing with respect to the image data received from the endoscope 2, generates an image, and outputs the image to the display 5. Moreover, the image processor 41 sets boundary regions determined based on the image, and calculates the time variation in the fluorescence intensity. The image processor 41 includes a boundary region calculator 411, a fluorescence intensity variation calculator 412, and a display image generator 413.

The boundary region calculator 411 determines, based on an image (a tissue structure image) that is generated based on the imaging signals generated by the first imaging element 244a and that is formed using the narrow band light, the boundary between a portion in which the tissue structure has changed and a portion in which the tissue structure either has not changes or has changed only slightly. As a result of determining a boundary, the boundary region calculator 411 determines a boundary region between a portion in which the tissue structure has changed and a portion in which the tissue structure either has not changed or has changed only slightly.

The fluorescence intensity variation calculator 412 calculates, for each boundary region, the time variation in the fluorescence intensity based on a second-type image that is based on an image generated by the second imaging element 244b and that is formed using the fluorescence light.

The display image generator 413 performs predetermined image processing and generates an image. Herein, an image can imply an image formed using the white light or the narrow band light; or an image indicating a boundary determined by the boundary region calculator 411; or an image corresponding to the variation calculated by the fluorescence intensity variation calculator 412; or an image in which visual information is attached to the fluorescence intensity itself. The predetermined image processing indicates synchronization, gray level correction, or color correction. The synchronization represents the operation of achieving synchronization among the image data of the RGB color components. The gray level correction represents the operation of correcting the gray level of the image data. The color correction represents the operation of performing color compensation with respect to the image data. Meanwhile, the display image generator 413 can also perform gain adjustment according to the brightness of an image.

The image processor 41 is configured either using a general-purpose processor such as a processing unit (CPU) or using a dedicated processor such as one of various arithmetic circuits, such as an application specific integrated circuit (ASIC), that implements specific functions. Moreover, the image processor 41 can be configured to include a frame memory for storing R image data, G image data, and B image data.

The synchronization signal generator 42 generates clock signals (synchronization signals) serving as the basis for the operations performed by the processing device 4, and outputs the generated synchronization signals to the light source device 3, the image processor 41, the controller 44, and the endoscope 2. Herein, the synchronization signals generated by the synchronization signal generator 42 include a horizontal synchronization signal and a vertical synchronization signal.

Thus, the light source device 3, the image processor 41, the controller 44, and the endoscope 2 perform operations in synchronization with each other based on the generated synchronization signals.

The input portion 43 is configured using a keyboard, a mouse, switches, or a touch-sensitive panel, and receives input of various signals such as an operation instruction signal that is meant for instructing the operations of the endoscope system 1. Meanwhile, the input portion 43 can also represent switches installed in the operating portion 22, or can be a portable terminal such as an external tablet computer.

The controller 44 performs driving control of the constituent elements including the imaging element 244 and the light source device 3, and performs input-output control of information with respect to the constituent elements. The controller 44 refers to control information data (for example, the reading timing) that is stored in the storage 45 and that is to be used in performing image control, and sends the control information data as driving signals to the imaging element 244 via predetermined signal lines included in the cable assembly 245. Moreover, the controller 44 switches between a normal observation mode meant for observing the images obtained in white light illumination and a fluorescence observation mode meant for calculating the fluorescence intensity of the excitation target. The controller 44 is configured using a general-purpose processor such as a CPU or using a dedicated processor such as one of various arithmetic circuits, such as an ASIC, that implements specific functions.

The storage 45 is used to store various computer programs meant for causing the endoscope system 1 to perform operations, and to store data containing various parameters required in the operations performed by the endoscope system 1. Moreover, the storage 45 is used to store the identification information of the processing device 4, which contains the specific information (ID), the model year, and the specifications information of the processing device 4.

Moreover, the storage 45 is used to store various computer programs including an image obtaining program that is meant for enabling the processing device 4 to implement an image obtaining method. The computer programs can be recorded for circulation in a computer-readable recording medium such as a hard disk, a flash memory, a CD-ROM, a DVD-ROM, or a flexible disk. Alternatively, the computer programs can be downloaded via a communication network, which is implemented using, for example, an existing public line, or a local area network (LAN), or a wide area network (WAN); and which can be a wired network or a wireless network.

The storage 45 is implemented using a read only memory (ROM) in which various computer programs are installed in advance, and using a random access memory (RAM) or a hard disk in which various operation parameters and data are stored.

The display 5 displays a display image corresponding to the image signal received from the processing device 4 (the image processor 41) via a video cable. The display 5 is configured using a monitor such as a liquid crystal display or an organic electroluminescence (EL) display.

The treatment tool device 6 includes a treatment tool operating portion 61, and includes a flexible treatment tool 62 that extends from the treatment tool operating portion 61. The treatment tool 62 that is used in photoimmunotherapy emits a light for enabling treatment (hereinafter, called the treatment light). The treatment tool operating portion 61 controls the emission of the treatment light from the treatment tool 62. The treatment tool operating portion 61 includes an operation input portion 611 that is configured using, for example, switches. In response to an input (for example, in response to the pressing of a switch) with respect to the operation input portion 611, the treatment tool operating portion 61 causes the treatment tool 62 to emit the treatment light. Meanwhile, in the treatment tool device 6, the light source that emits the treatment light either can be installed in the treatment tool 62 or can be installed in the treatment tool operating portion 61. The light source is implemented using a semiconductor laser or a light emitting diode (LED). For example, in the case of implementing photoimmunotherapy, the treatment light has the wavelength band equal to or greater than 680 nm and, for example, has the central wavelength of 690 nm (for example, the light L_{P} illustrated in FIG. 5).

Herein, the illumination optical system included in the treatment tool 62 can be configured to change the application range of the treatment light. For example, under the control of the treatment tool operating portion 61, the illumination optical system can be configured either using an optical system in which the focal distance can be varied or using a digital micromirror device (DMD); and it is possible to vary the spot diameter of the light applied onto the subject and to vary the shape of the application range.

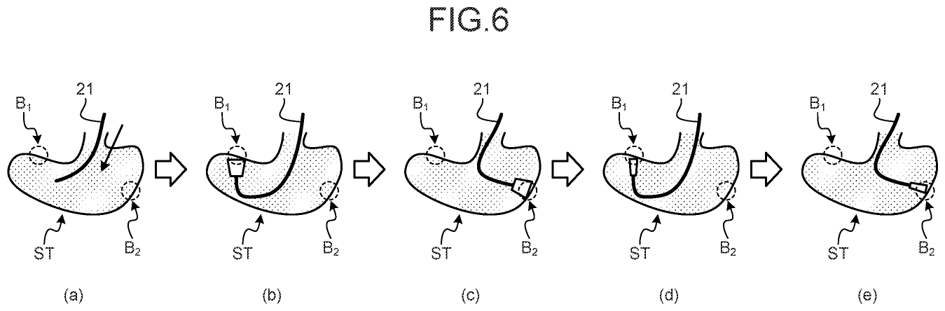

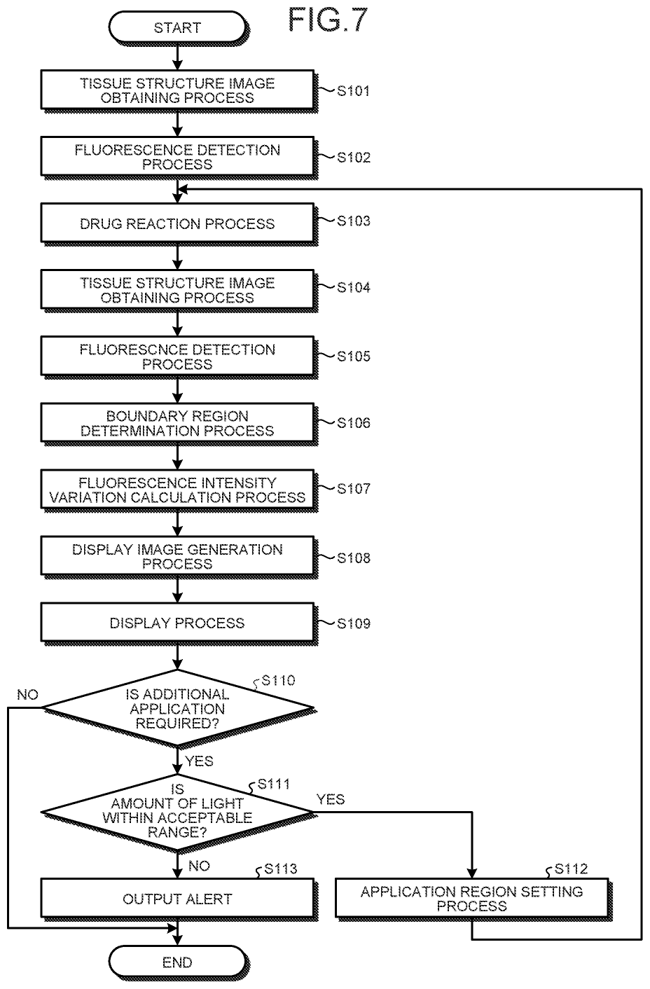

Explained below with reference to FIGS. 6 and 7 is the flow of the treatment performed using the endoscope 2. FIG. 6 is a diagram illustrating an exemplary flow of the treatment performed using the endoscope according to the first embodiment of the disclosure. In FIG. 6 is illustrated an example of implementing photoimmunotherapy; and the insertion portion 21 is inserted into a stomach ST for carrying out the treatment.

Firstly, the operator inserts the insertion portion 21 into the stomach ST (see (a) in FIG. 6). At that time, the operator instructs the light source device 3 to emit the white light and, while observing the white-light image that captures the inside of the stomach ST and that is displayed in the display 5, searches for the treatment position. Herein, it is assumed that the treatment is carried out for tumors B₁ and B₂ representing the treatment targets. The operator observes the white-light image and decides on the regions that include the tumors B₁ and B₂ as application regions.

The operator orients the front end portion 24 toward the tumor B₁, projects the treatment tool 62 from the front end of the endoscope 2, and applies the treatment light onto the tumor B₁ (see (b) in FIG. 6). As a result of the application of the treatment light, the antibody drug that is bound to the tumor B₁ reacts, and the treatment of the tumor B₁ is carried out.

Then, the operator orients the front end portion 24 toward the tumor B₂, projects the treatment tool 62 from the front end of the endoscope 2, and applies the treatment light onto the tumor B₂ (see (c) in FIG. 6). As a result of the application of the treatment light, the antibody drug that is bound to the tumor B₂ reacts, and the treatment of the tumor B₂ is carried out.

Subsequently, the operator orients the front end portion 24 toward the tumor B₁ and applies the excitation light onto the tumor B₁ from the front end of the endoscope 2 (see (d) in FIG. 6). Then, the operator observes the fluorescence intensity and confirms the effect of treatment on the tumor B₁. As far as confirming the effect of treatment is concerned, the operator makes the determination based on the image display (explained later).

Subsequently, the operator orients the front end portion 24 toward the tumor B₂ and applies the excitation light onto the tumor B₂ from the front end of the endoscope 2 (see (e) in FIG. 6). Then, the operator observes the fluorescence intensity and confirms the effect of treatment on the tumor B₂.

Herein, as may be necessary, the operator again applies the treatment light and confirms the effect of treatment in a repeated manner.

Explained below with reference to FIG. 7 are the operations performed by the processing device 4. FIG. 7 is a flowchart for explaining an example of the operations performed by the processing device according to the first embodiment. In an identical manner to FIG. 6, in FIG. 7 is illustrated an exemplary flow at the time of implementing photoimmunotherapy.

Firstly, before the application of the treatment light, the narrow band light is applied onto the treatment position from the front end portion 24, and a pre-treatment tissue structure image is obtained (Step S101: tissue structure image obtaining process). In the processing device 4, a tissue structure image is generated based on the imaging signal generated by the first imaging element 244a.

Then, the light source device 3 is made to emit the excitation light, and the fluorescence of the antibody drug is detected (Step S102: fluorescence detection process). When emitted, the excitation light is applied onto the photographic subject from the endoscope 2, and the pre-treatment antibody drug gets excited and emits fluorescence. At that time, the processing device 4 obtains the imaging signal (fluorescence image) generated by the second imaging element 244b.

Subsequently, in response to an operation of the operator, the treatment light is applied from the treatment tool 62 onto the antibody drug that is bound to the cancer cells, thereby resulting in the reaction of the antibody drug (Step S103: drug reaction process). During the drug reaction process, the treatment is carried out in which the antibody drug gets activated as a result of the application of the near-infrared light representing the treatment light, and the cancer cells are destroyed.

Then, the narrow band light is applied from the front end portion 24 onto the treatment position, and a post-treatment tissue structure image is obtained (Step S104: tissue structure image obtaining process). At Step S104 too, in an identical manner to Step S101, the processing device 4 generates a tissue structure image based on the imaging signal generated by the first imaging element 244a.

Subsequently, the light source device 3 is made to emit the excitation light, and the fluorescence of the antibody drug is detected (Step S105: fluorescence detection process). At Step S105 too, in an identical manner to Step S102, the processing device 4 obtains the imaging signal (fluorescence image) generated by the second imaging element 244b.

The boundary region calculator 411 uses the tissue structure images obtained at Steps S101 and S103, and determines the boundary regions by determining the boundaries between the regions having a fast reaction speed and the regions having a slow reaction speed (Step S106: boundary region determination process). Meanwhile, the boundary region determination process either can be performed before the fluorescence detection process or can be performed simultaneous to the fluorescence detection process.

Given below is the explanation of a determination operation performed by the boundary region calculator 411. For example, the boundary region calculator 411 performs either a first determination operation or a second determination operation explained below, and determines a boundary region. However, the boundary region determination can also be performed using some other known method other than the first and second determination operations.

### First Determination Operation

The boundary region calculator 411 detects the time variation in two tissue structure images obtained at different timings; and accordingly determines, as a boundary region, a region in which the body part exhibits a change in the tissue structure and whose outer edge represents the boundary. For example, the boundary region calculator 411 compares the value (luminance value) of a tissue structure image with a preset threshold value; extracts a region of the body part exhibiting a change in the tissue structure; and determines, as the boundary region, the extracted region whose outer edge represents the boundary. The threshold value can be a preset luminance value in the normal state (i.e., the state without having any tumor), or can be the luminance value of a tissue structure image obtained before the treatment.

### Second Determination Operation

The boundary region calculator 411 uses a feature calculated in advance according to machine learning and determines, as the boundary region, the region in which the body part exhibits a change in the tissue structure. The boundary region calculator 411 calculates the feature of a tissue structure image that is obtained, and determines the boundary region using the calculated feature and a learning model.

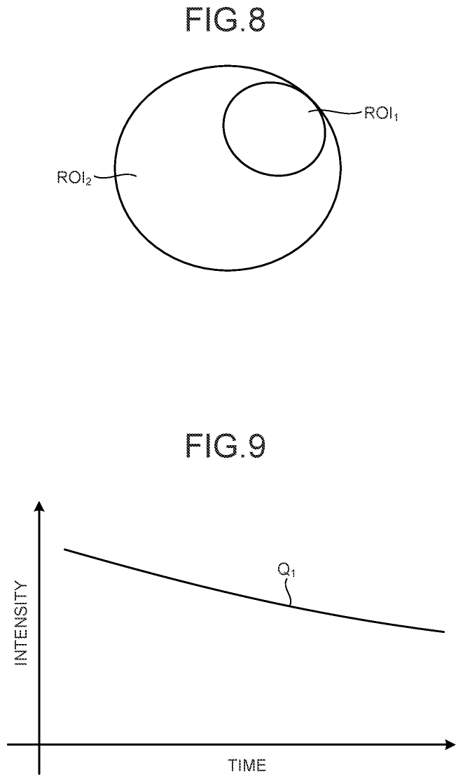

FIG. 8 is a diagram for explaining about the regions separated according to boundary region determination. The boundary region calculator 411 compares tissue structure images; detects the boundary between a region exhibiting significant changes in the tissue, which represents the region having a fast reaction speed, and a region exhibiting only a small change in the tissue, which represents the region having a slow reaction speed; and determines the boundary region. For example, the boundary region calculator 411 sets a first region ROI₁ as the region having a slow reaction speed, and sets a second region ROI₂ as the region having a fast reaction speed.

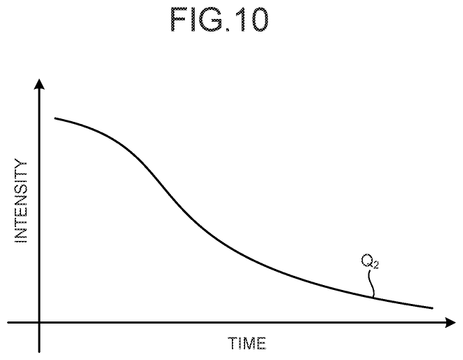

FIG. 9 is a diagram illustrating an exemplary transition of the fluorescence intensity when the reaction progress speed is slow. FIG. 10 is a diagram illustrating an exemplary transition of the fluorescence intensity when the reaction progress speed is fast. In the region having a slow reaction speed (for example, the first region ROIL), there is a low attenuation rate of a fluorescence intensity Q: attributed to the antibody drug, and a high intensity is maintained over time (see FIG. 9). On the other hand, in the region having a fast reaction speed (for example, the second region ROI), there is a high attenuation rate of a fluorescence intensity Q₂ attributed to the antibody drug (see FIG. 10).

The fluorescence intensity variation calculator 412 calculates the fluorescence intensity variation using the fluorescence images obtained at Steps S102 and S105 (Step S107: fluorescence intensity variation calculation process). For each boundary region determined by the boundary region calculator 411, the fluorescence intensity variation calculator 412 calculates the variation in the fluorescence intensity (the difference value between the pre-treatment fluorescence intensity and the post-treatment fluorescence intensity). Meanwhile, at that time, using a known method such as pattern matching, the positioning of the pre-treatment image and the post-treatment image can also be performed.

Then, the display image generator 413 generates an image to be displayed in the display 5 (Step S108). The display image generator 413 generates an image in which the variation in the fluorescence intensity is visually expressed. For example, the display image generator 413 generates an image by superimposing visual information, which corresponds to the variation in the fluorescence intensity, onto a tissue structure image; or generates an image by superimposing visual information, which corresponds to the time variation in the fluorescence intensity (i.e., the fluorescence intensity variation) in each boundary region, and superimposing the boundary line of a boundary region (for example, the first region ROIL) onto a tissue structure image; or generates an image in which the time variation of the fluorescence intensity of each boundary region (for example, refer to FIGS. 9 and 10) is displayed along with an image. As the visual information corresponding to the fluorescence intensity, for example, a region exhibiting only a small variation in the fluorescence intensity is given an easily-recognizable color (i.e., an easily-identifiable hue or an easily-identifiable color density for a human eye). Using the display image, for example, the difference in the fluorescence intensity variation of mutually different boundary regions (for example, the first region ROI₁ and the second region ROI₂) can be made easily recognizable. Meanwhile, the display image generator 413 can generate an image including only the tissue structure, or can generate a white light image, or can generate a fluorescence intensity image (intensity map).

The controller 44 displays the image, which is generated at Step S108, in the display 5 (Step S109: display process). As a result of displaying the image in the display 5, the operator is asked to confirm the effect of treatment. The operator looks at the image and confirms the effect of treatment, and accordingly determines whether or not to again apply the treatment light and determines the region for applying the treatment light (for example, the first region ROI₁). Then, the operator operates the input portion 43 and inputs the determination result.

When the input portion 43 receives input of the determination result, the controller 44 determines whether or not an additional application of the treatment light is to be performed (Step S110). Based on the input determination result, if it is determined that the additional application of the treatment light is not required (No at Step S110), then the operations are ended. On the other hand, if it is determined that the additional application of the treatment light is required (Yes at Step S110), then the system control proceeds to Step S111.

In the case of performing the additional application of the treatment light, for example, in the illumination optical system, control is performed to match the shape of the light application range to the shape of the boundary region, and the operator adjusts the spot diameter and applies the treatment light.

The controller 44 determines whether or not, in the region on which the additional application of the treatment light is to be performed, the amount of already-applied light is within the acceptable range (Step S111). The acceptable range represents a preset amount of light and is set to have at least the upper limit value. The upper limit value is set so as to hold down any damage to the tissue due to excessive application of the treatment light. Thus, for example, the controller 44 determines whether or not the amount of light already applied to the target region (i.e., the cumulative amount of light) is exceeding the upper limit value.

If the controller 44 determines that the amount of light is within the acceptable range (i.e., smaller than the upper limit value) (Yes at Step S111), then the system control proceeds to Step S112. On the other hand, if the controller 44 determines that the amount of already-applied light is outside the acceptable range (i.e., is exceeding the upper limit value) (No at Step S111), then the system control proceeds to Step S113.

At Step S112, the controller 44 sets an application region for additional application of the treatment light. After the controller 44 sets the application region, the system control returns to Step S103 and the operations are repeated.

At Step S113, the controller 44 outputs an alert indicating that the amount of applied light has exceeded the acceptable range. The alert can be displayed as character information in the display 5, or can be issued in the form of a sound or a light, or can be a combination thereof. After the alert is displayed in the display 5, the controller 44 ends the operations.

In the first embodiment described above, a tissue structure image is obtained using the narrow band light; the regions having different reaction speeds (the boundary regions) are separated according to the changes occurring in the tissue before and after the treatment; and the variation in the fluorescence intensity of each region is calculated. At that time, either the boundary regions are displayed or the variation in the fluorescence intensity in each boundary region is displayed, and the operator is asked to determine whether or not the additional application of the treatment light is to be performed in each boundary region. According to the first embodiment, since the additional application of the treatment light can be ensured on a region-by-region basis, the application of the treatment light with respect to the treatment region can be carried out in an appropriate manner.

Moreover, in the first embodiment, after the effect of treatment is confirmed according to the fluorescence, at the time of additional application of the treatment light, the cumulative amount of treatment light applied to the concerned region is compared with the acceptable range. If the cumulative amount of treatment light has exceeded the acceptable range, then an alert is issued to indicate that the cumulative amount of treatment light has exceeded the acceptable range. Thus, according to the first embodiment, it becomes possible to hold down the damage to the tissue due to excessive application of the treatment light.

Meanwhile, in the first embodiment, the first imaging element 244a can be configured using a multi-band image sensor, so that the lights having a plurality of mutually different wavelength bands can be individually obtained. For example, the scattering light or the returning light of the light having the wavelength band equal to or greater than 380 nm and equal to or smaller than 440 nm, and the scattering light or the returning light of the light having the wavelength band equal to or greater than 490 nm and equal to or smaller than 590 nm can be individually obtained using a multi-band image sensor; and a narrow band light image corresponding to each light can be generated. As a result, it becomes possible to obtain blood vessel images having different depths from the superficial portion of the mucous membrane, and to determine the boundary regions with a higher degree of accuracy according to the changes occurring in the blood vessel and the tissue at each depth.

### Modification Example of First Embodiment

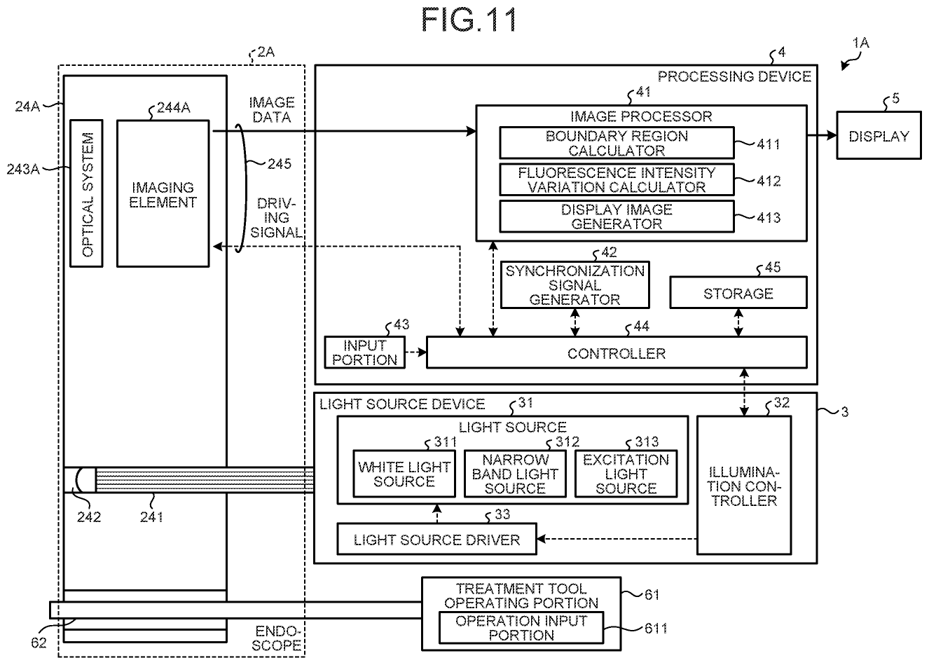

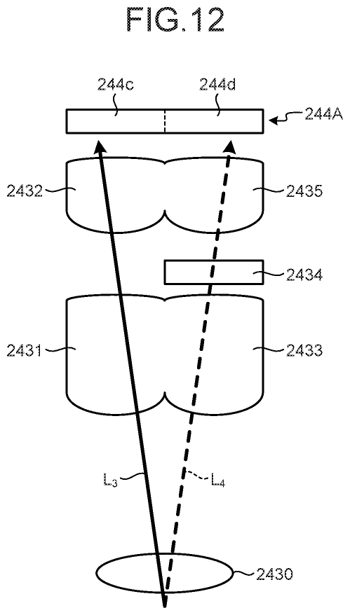

Explained below with reference to FIGS. 11 and 12 is a modification example of the first embodiment. FIG. 11 is a block diagram illustrating an overall configuration of an endoscope system according to the modification example of the first embodiment. An endoscope system 1A according to the modification example includes an endoscope 2A in place of the endoscope 2 of the endoscope system 1 according to the first embodiment. Other than the endoscope 2A, the configuration is same as the first embodiment. Hence, the same explanation is not given again.

The endoscope 2A includes a front end portion 24A in place of the front end portion 24 of the endoscope 2. Other than the front end portion 24A, the configuration is same as the endoscope 2. Hence, the same explanation is not given again.

The front end portion 24A includes the light guide 241; the illumination lens 242; an optical system 243A that collects light; and an imaging element 244A that is disposed at the image formation position of the optical system 243A and that receives the light collected by the optical system 243A, performs photoelectric conversion, and performs predetermined signal processing with respect to electrical signals.

FIG. 12 is a diagram for explaining a configuration of an imaging optical system of the endoscope according to the modification example of the first embodiment of the disclosure. The optical system 243A and the imaging element 244A are installed inside the front end portion 24A.

The optical system 243A includes an objective lens 2430; a first lens 2431 made of one or more optical elements; a second lens 2432 made of one or more optical elements; a third lens 2433 made of one or more optical elements; a cutoff filter 2434; and a fourth lens 2435 made of one or more optical elements. The cutoff filter 2434 cuts off the light having the wavelength band of the excitation light. Herein, the excitation light represents the light having the wavelength band meant for causing excitation of the antibody drug during photoimmunotherapy. The second lens 2432 and the fourth lens 2435 form observation images at mutually different and nonoverlapping positions on the imaging element 244A.

The transmittance of the excitation light through the cutoff filter 2434 is, for example, set to be equal to lower than 0.1%. As a result of setting the transmittance of the excitation light to be equal to lower than 0.1%, the fluorescence can be selectively incorporated at the time of excitation light illumination.

The imaging element 244A performs photoelectric conversion of the light coming from the optical system 243A and generates an electrical signal (an image signal). More particularly, in the imaging element 244A, a plurality of pixels, each of which includes a photodiode for storing the electrical charge according to the amount of light and includes a capacitor for converting the electrical charge transferred from the photodiode into a voltage level, is arranged as a two-dimensional matrix. Each pixel performs photoelectric conversion with respect to the light coming from the optical system 243A and generates an electrical signal, and outputs the electrical signal as an image signal. The imaging element 244A is configured using, for example, a CCD image sensor or a CMOS image sensor.

Via the objective lens 2430, lights L₃ and L₄ coming from the photographic subject fall on the first lens 2431 and the third lens 2433, respectively. The light L₃ that falls on the first lens 2431 gets converted into an image by the second lens 2432. The light L₄ that falls on the third lens 2433 passes through the cutoff filter 2434 and gets converted into an image by the fourth lens 2435.

The second lens 2432 forms an observation image in a first imaging portion 244c of the imaging element 244A. The fourth lens 2435 forms an observation image in a second imaging portion 244d of the imaging element 244A. The first imaging portion 244c and the second imaging portion 244d are formed by dividing the light receiving region of the imaging element 244 into two portions.

In the case of implementing photoimmunotherapy, the processing device 4 performs operations according to the flow illustrated in FIG. 7. At that time, the first imaging element 244a is loaded in the first imaging portion 244c, and the second imaging element 244b is loaded in the second imaging portion 244d.

In the modification example explained above, in an identical manner to the first embodiment, a tissue structure image is obtained using the narrow band light; the regions having different reaction speeds (the boundary regions) are separated according to the changes occurring in the tissue before and after the treatment; and the variation in the fluorescence intensity of each region is calculated. At that time, either the boundary regions are displayed or the variation in the fluorescence intensity in each boundary region is displayed, and the operator is asked to determine whether or not the additional application of the treatment light is to be performed in each boundary region. According to the modification example, since the additional application of the treatment light can be ensured on a region-by-region basis, the application of the treatment light with respect to the treatment region can be carried out in an appropriate manner.

### Second Embodiment

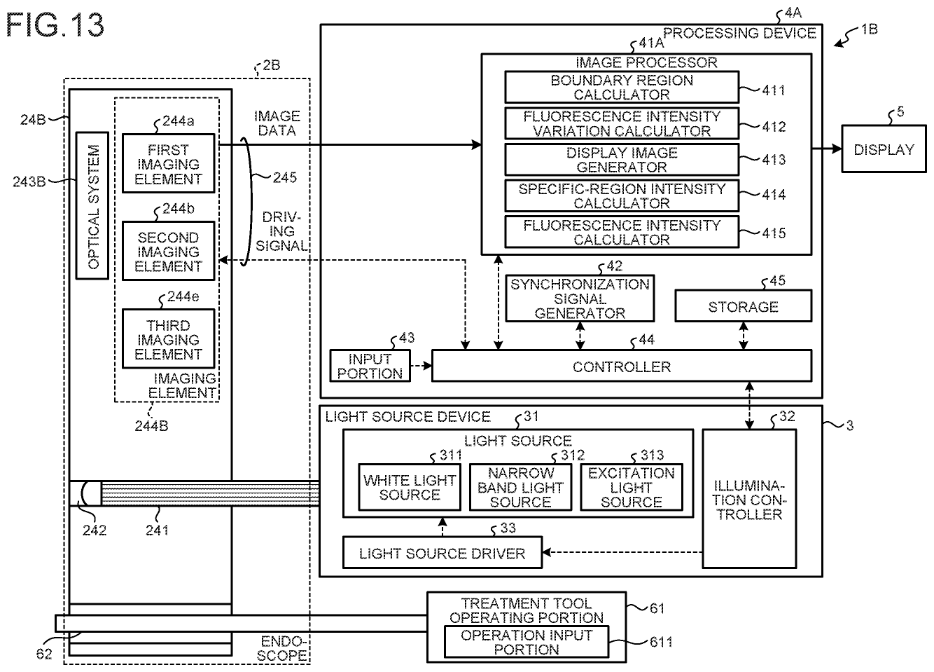

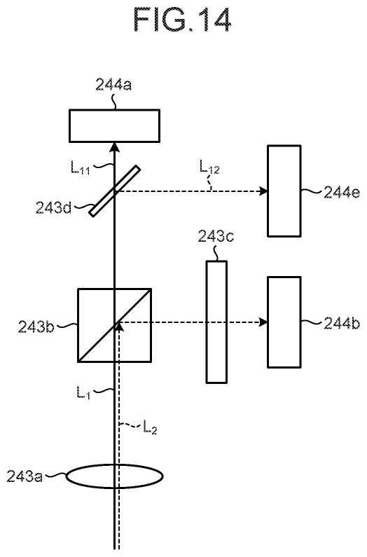

A second embodiment is described below with reference to FIGS. 13 and 14. FIG. 13 is a diagram illustrating an overall configuration of an endoscope system according to the second embodiment of the disclosure. An endoscope system 1B according to the second embodiment includes an endoscope 2B and a processing device 4A in place of the endoscope 2 and the processing device 4, respectively, of the endoscope system 1 according to the first embodiment. Other than the endoscope 2A and the processing device 4A, the configuration is same as the first embodiment. Hence, the same explanation is not given again.

The endoscope 2B includes a front end portion 24B in place of the front end portion 24 of the endoscope 2. Other than the front end portion 24B, the configuration is same as the endoscope 2. Hence, the same explanation is not given again.

The front end portion 24B includes the light guide 241; the illumination lens 242; an optical system 243B that collects light; and an imaging element 244A that is disposed at the image formation position of the optical system 243B and that receives the light collected by the optical system 243B, performs photoelectric conversion, and performs predetermined signal processing with respect to electrical signals.

FIG. 14 is a diagram for explaining a configuration of an imaging optical system of the endoscope according to the second embodiment of the disclosure. The optical system 243B and an imaging element 244B are installed inside the front end portion 24B.

The optical system 243B includes the objective lens 243a; a dichroic mirror 243b (hereinafter, referred to as “first dichroic mirror 243b”); the cutoff filter 243c, and a second dichroic mirror 243d. The cutoff filter 243c cuts off the light having the wavelength band of the excitation light. The second dichroic mirror 243d bends the light path of the light having the wavelength band of the blue component, such as the light having the wavelength band equal to or smaller than 490 nm, and lets the light having the wavelength band of the other components (for example, the green component and the red component) pass through. Apart from including the optical elements mentioned above, the optical system 243B can also include lenses.

The light coming from the photographic subject falls on the first dichroic mirror 243b via the objective lens 243a. The first dichroic mirror 243b bends the light path of the light having the wavelength equal to or greater than the wavelength of the fluorescence (i.e., bends a light L₂), and lets the light having the wavelength smaller than the wavelength of the fluorescence pass through (i.e., lets a light L: pass through). The light that has passed through the first dichroic mirror (i.e., the light L₁) falls on the second dichroic mirror 243d. On the other hand, regarding the excitation light and the fluorescence (i.e., the light L₂) having the light path bent by the first dichroic mirror 243b; the excitation light is cut off by the cutoff filter 243c, and the fluorescence falls on the second imaging element 244b.

The second dichroic mirror 243d bends the light path of the light that includes the returning light of the narrow band light having the wavelength band equal to or greater than 440 nm and equal to or smaller than 490 nm (i.e., bends a light L₁₂), and lets the light of the color components other than the blue component (for example, the components having the wavelength greater than 490 nm) pass through (i.e., lets a light L₁a pass through). The light that has passed through the second dichroic mirror 243d (i.e., the light L₁₁) falls on the first imaging element 244a. On the other hand, the light having the light path bent by the second dichroic mirror 243d (i.e., the light L₁₂) falls on a third imaging element 244e.

The imaging element 244B performs photoelectric conversion of the light coming from the optical system 243B and generates an electrical signal (an image signal). More particularly, the imaging element 244B includes three imaging elements (the first imaging element 244a, the second imaging element 244b, and the third imaging element 244e). Each of the first imaging element 244a to the third imaging element 244e are configured using, for example, a CCD image sensor or a CMOS image sensor.

Given below is the explanation of a configuration of the processing device 4A. The processing device 4A includes an image processor 41A, the synchronization signal generator 42, the input portion 43, the controller 44, and the storage 45.

The image processor 41A receives, from the endoscope 2B, image data of the illumination light of each color as obtained by the imaging element 244B by performing imaging. Then, the image processor 41A performs predetermined image processing with respect to the image data received from the endoscope 2B, generates an image, and outputs the image to the display 5. Moreover, the image processor 41A sets the boundary regions determined based on the image, and calculates the time variation in the fluorescence intensity. The image processor 41A includes the boundary region calculator 411, the fluorescence intensity variation calculator 412, the display image generator 413, a specific-region intensity calculator 414, and a fluorescence intensity calculator 415.

In the second embodiment, the display image generator 413 generates a white light image based on the electrical signals generated by the first imaging element 244a and the third imaging element 244e.

The specific-region intensity calculator 414 calculates the light intensity of a specific wavelength band. In the second embodiment, the specific-region intensity calculator 414 calculates the light intensity of the light having the wavelength band of the blue component (i.e., the light L₁₂). Herein, the specific-region intensity calculator 414 calculates the light intensity of the blue component based on the electrical signal generated by the third imaging element 244e.

The fluorescence intensity calculator 415 divides the intensity variation, which is calculated by the fluorescence intensity variation calculator 412, by the light intensity of the blue component as calculated by the specific-region intensity calculator 414; and standardizes the intensity variation.

In the case of implementing photoimmunotherapy, the processing device 4A performs operations according to the flow illustrated in FIG. 7. At that time, during the fluorescence detection process (Step S105), the excitation light is applied onto the photographic subject along with applying the narrow band light having the wavelength band equal to or greater than 440 nm and equal to or smaller than 490 nm. As a result, the specific-region intensity calculator 414 can calculate the light intensity of the returning light of the narrow band light having the wavelength band equal to or greater than 440 nm and equal to or smaller than 490 nm. Meanwhile, instead of applying it during the fluorescence detection process, the narrow band light can alternatively be applied at a different timing.

During the fluorescence intensity variation calculation process (Step S107), the fluorescence intensity variation, which has been standardized by the fluorescence intensity calculator 415, is calculated. Meanwhile, during the boundary region determination process (Step S106), the boundary region calculator 411 can determine the boundary regions either based on the electrical signal generated by the first imaging element 244a, or based on the electrical signal generated by the third imaging element 244e, or based on the electrical signals generated by the first imaging element 244a and the third imaging element 244e.

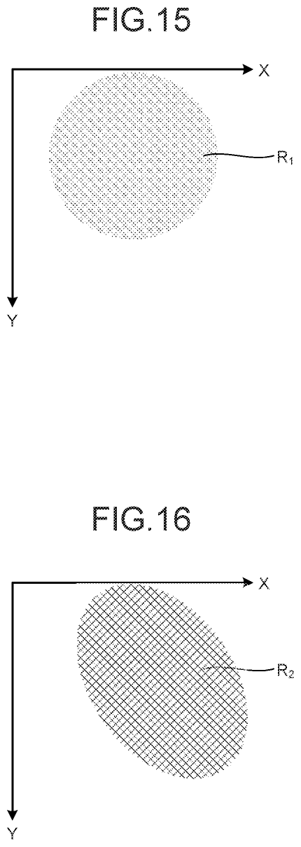

Regarding the boundary region determination, given below is the explanation with reference to FIGS. 15 to 17. FIG. 15 is a diagram that schematically illustrates an image obtained by the first imaging element. FIG. 16 is a diagram that schematically illustrates an image obtained by the third imaging element.

The image obtained by the first imaging element 244a is based on an image formed using the light having the wavelength band that excludes the wavelength band of the fluorescence component and the blue component. The image obtained by the third imaging element 244e is based on an image formed using the light having the wavelength band of the blue component. For example, assume that the first imaging element 244a obtains the image illustrated in FIG. 15 and that the third imaging element 244e obtains the image illustrated in FIG. 16. In FIGS. 15 and 16, the X-axis and the Y-axis are illustrated to indicate the relative positional relationship of the images. The images illustrated in FIGS. 15 and 16 are based on the lights having mutually different wavelength bands (i.e., the wavelength band of the blue component, and the wavelength band excluding the wavelength band of the blue component and the fluorescence), and different tissue structures are visualized therein. More particularly, blood vessels having mutually different depths from the tissue surface are visualized. In FIGS. 15 and 16, images of the tissue structure are visualized in light detection regions R₁ and R₂, respectively.

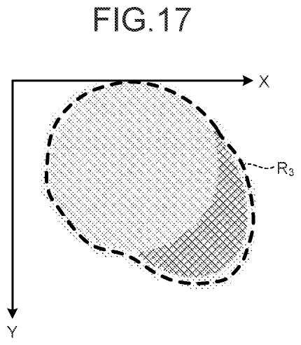

Based on the image obtained by the first imaging element 244a (for example, the image illustrated in FIG. 15; hereinafter, called a first image) and based on the image obtained by the third imaging element 244e (for example, the image illustrated in FIG. 16; hereinafter, called a second image), the boundary region calculator 411 determines the boundary regions having different degrees of variation in the tissue structure. FIG. 17 is a diagram for explaining the boundary region set as a result of combining the image illustrated in FIG. 15 and the image illustrated in FIG. 16. The boundary region calculator 411 synthesizes the first image and the second image; extracts the contour of the synthesized image; and the treats the extracted contour as the boundary region. In FIG. 17, a dashed line R₃ is set as the boundary region.

In the second embodiment described above, in an identical manner to the first embodiment, a tissue structure image is obtained using the narrow band light; the regions having different reaction speeds (the boundary regions) are separated according to the changes occurring in the tissue before and after the treatment; and the variation in the fluorescence intensity of each region is calculated. At that time, either the boundary regions are displayed or the variation in the fluorescence intensity in each boundary region is displayed, and the operator is asked to determine whether or not the additional application of the treatment light is to be performed in each boundary region. According to the second embodiment, since the additional application of the treatment light can be ensured on a region-by-region basis, the application of the treatment light with respect to the treatment region can be carried out in an appropriate manner.

Moreover, in the second embodiment, since the intensity variation of the fluorescence is standardized, when the standardized fluorescence intensity variation is displayed, it can be ensured that the operator appropriately understands the fluorescence intensity variation regardless of the distance between the endoscope 2B (the front end portion 24B) and the photographic subject. Meanwhile, the narrow band obtained for the standardization purpose is not limited to the wavelength band equal to or greater than 400 nm and equal to or smaller than 490 nm, and some other wavelength band can also be obtained. The light having the wavelength band equal to or greater than 400 nm and equal to or smaller than 490 nm does not have any contribution from the absorption attributed to the blood component, and the scattering light coming from the body tissue remains the dominant factor. Hence, the intensity of the scattering light coming from the tissue is dependent only on the distance, thereby making it suitable to cancel the distance-attributable fluctuation in the division-based fluorescence intensity.

### Third Embodiment

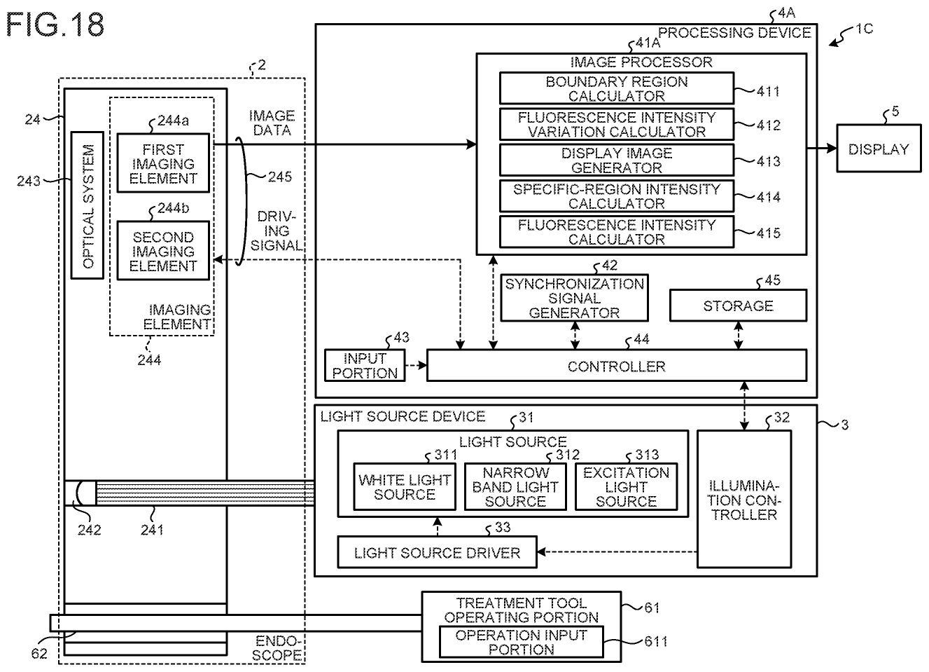

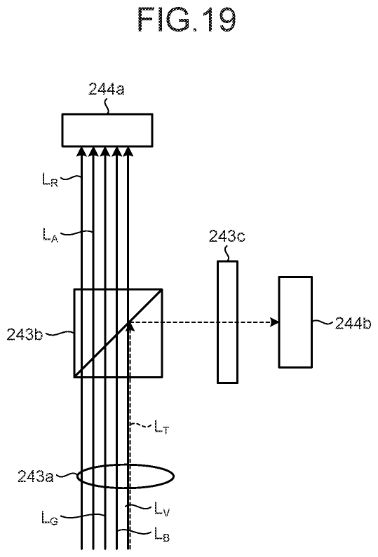

A third embodiment is described below with reference to FIGS. 18 and 19. FIG. 18 is a diagram illustrating an overall configuration of an endoscope system according to the third embodiment of the disclosure. An endoscope system 1C according to the third embodiment includes the processing device 4A in place of the processing device 4 of the endoscope system 1 according to the first embodiment. Moreover, the front end portion 24 includes the optical system 243 and the imaging element 244 in an identical manner to the first embodiment. However, the first imaging element 244a is configured using a multi-band image sensor that generates an electrical signal on an individual basis for each color component.

FIG. 19 is a diagram for explaining a configuration of an imaging optical system of the endoscope according to the third embodiment of the disclosure. For example, the light that reflects or scatters from the photographic subject includes the following lights: the narrow band light L_{R} having the central wavelength of 660 nm; the light L_{A} having the central wavelength of 590 nm; the light L_{C} having the central wavelength of 525 nm; the light L_{B} having the central wavelength of 480 nm; the light L_{V} having the central wavelength of 380 nm; the excitation light (for example, the light L_{P} illustrated in FIG. 5); and a light L_{T} including the fluorescence excited due to the excitation light. The light L_{T} falls on the second imaging element 244b after the excitation light gets cut off by the cutoff filter 243c.

The lights L_{R}, L_{A}, L_{G}, L_{B}, and L_{V} that have passed through the dichroic mirror 243b further pass through various filters and individually fall onto the first imaging element 244a. Then, the first imaging element 244a performs individual photoelectric conversion of the lights L_{R}, L_{A}, L_{G}, L_{B}, and L_{V}; and generates electrical signals.

In the third embodiment, the specific-region intensity calculator 414 calculates the light intensity using the electrical signal, from among the electrical signals generated by the first imaging element 244a, which is generated based on the light of the blue component (i.e., the light L_{B}).

In the case of implementing photoimmunotherapy, the processing device 4A performs operations according to the flow illustrated in FIG. 7. At that time, during the fluorescence intensity variation calculation process (Step S107), the fluorescence intensity variation, which has been standardized by the fluorescence intensity calculator 415, is calculated. Meanwhile, during the boundary region determination process (Step S106), the boundary region calculator 411 can determine the boundary regions either based on the electrical signal generated by the first imaging element 244a, or based on the electrical signal corresponding to the light of the blue component, or based on the electrical signals corresponding to the lights of the components other than the blue component, or based on the electrical signals of all color components as generated by the first imaging element 244a. Herein, the electrical signals of all color components represent the electrical signals that are generated by a plurality of filters included in the multi-band image sensor and that have mutually different wavelength bands for receiving a light or letting a light pass through.

In the third embodiment described above, in an identical manner to the first embodiment, a tissue structure image is obtained using the narrow band light; the regions having different reaction speeds (the boundary regions) are separated according to the changes occurring in the tissue before and after the treatment; and the variation in the fluorescence intensity of each region is calculated. At that time, either the boundary regions are displayed or the variation in the fluorescence intensity in each boundary region is displayed, and the operator is asked to determine whether or not the additional application of the treatment light is to be performed in each boundary region. According to the third embodiment, since the additional application of the treatment light can be ensured on a region-by-region basis, the application of the treatment light with respect to the treatment region can be carried out in an appropriate manner.

Meanwhile, in the third embodiment, the explanation is given about the case in which the first imaging element 244a individually generates the electrical signal for each color component. Alternatively, the first imaging element 244a can be configured to individually generate: an electrical signal based on the light equivalent to the returning light of the narrow band light having the wavelength band equal to or greater than 440 nm and equal to or smaller than 490 nm; and an electrical signal based on the lights of the components other than the returning light.

### Fourth Embodiment

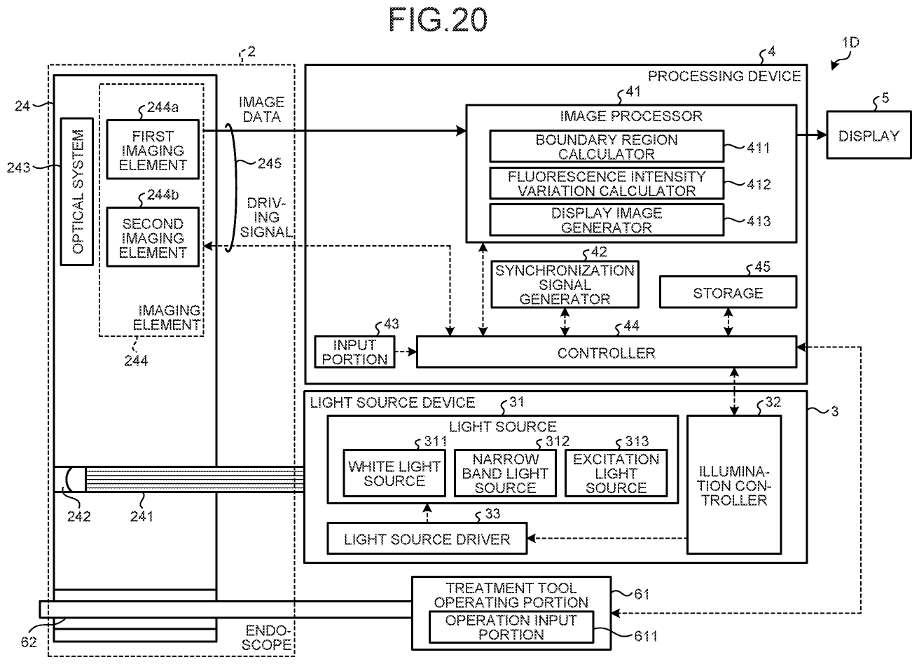

A fourth embodiment is described below with reference to FIG. 20. FIG. 20 is a diagram illustrating an overall configuration of an endoscope system according to the fourth embodiment of the disclosure. An endoscope system 1D according to the fourth embodiment has an identical configuration to the configuration of the endoscope system 1 according to the first embodiment. In the endoscope system 1D, the processing device 4 is electrically connected to the treatment tool device 6, and the controller 44 of the processing device 4 controls the emission of the treatment light from the treatment tool 62.

In the case of implementing photoimmunotherapy, the processing device 4 performs operations according to the flow illustrated in FIG. 7. At the time of applying the treatment light, the controller 44 controls the application range, the application timing, and the application period of the treatment light. More particularly, for example, with respect to the application range set by the operator, the controller 44 sets a light intensity (output value) representing a preset amount of applied light, and sets the application period. In response to the pressing of a switch of the operation input portion 611, the controller 44 starts the application control of the treatment light. At the time of additional application of the treatment light, the controller 44 sets the shape of the application range of the treatment light, which is emitted from the treatment tool 62, according to the target boundary region; and, in response to the pressing of a switch of the operation input portion 611, starts the application control of the treatment light. Meanwhile, the controller 44 can determine whether or not the cumulative amount of applied light in the target region for application has exceeded a preset upper limit value. If the upper limit value has been exceeded, the controller 44 can issue an alert.

In the fourth embodiment described above, in an identical manner to the first embodiment, a tissue structure image is obtained using the narrow band light; the regions having different reaction speeds (the boundary regions) are separated according to the changes occurring in the tissue before and after the treatment; and the variation in the fluorescence intensity of each region is calculated. At that time, either the boundary regions are displayed or the variation in the fluorescence intensity in each boundary region is displayed, and the operator is asked to determine whether or not the additional application of the treatment light is to be performed in each boundary region. According to the fourth embodiment, since the additional application of the treatment light can be ensured on a region-by-region basis, the application of the treatment light with respect to the treatment region can be carried out in an appropriate manner.

Moreover, in the fourth embodiment, since the controller 44 controls the emission of the treatment light from the treatment tool 62, the operator need not adjust the application range of the treatment light in accordance with the boundary region, and the treatment light can be applied onto an appropriate region.

In the embodiments described above, the excitation light and the treatment light either can have the same wavelength band (the same central wavelength) or can have mutually different wavelength bands (mutually different central wavelengths). Meanwhile, when the excitation light is used in common with the treatment light, it serves the purpose as long as the treatment light (the excitation light) is applied using the treatment tool 62 or the excitation light source 313. Hence, either the excitation light source 313 or the treatment tool 62 can be omitted from the configuration.

Moreover, in the embodiments described above, the explanation is given about the example in which the light source device 3 and the processing device 4 are separate devices. Alternatively, the light source device 3 and the processing device 4 can be integrated into a single device. Furthermore, in the embodiments described above, the explanation is given about the example in which the treatment light is applied using a treatment tool. Alternatively, the light source device 3 can be configured to emit the treatment light.

Moreover, in the embodiments described above, the endoscope system 1, which treats the body tissue inside a subject as the observation target and which includes the flexible endoscope 2, represents the endoscope system according to the disclosure. Alternatively, it is also possible to use an endoscope system in which a rigid endoscope is used, or an industrial endoscope is used for observing the characteristic features of materials, or a fiberscope is used, or such a device is used in which a camera head is connected to the eyepiece of an optical endoscope such as an optical viewing tube.

### NOTE

A phototherapy method including:
inserting a front end portion of an endoscope up to a target body part for treatment;
applying treatment light onto the target body part for treatment to cause a reaction of a drug which is bound to the target body part for treatment;
using a tissue structure image which is formed using narrow band light applied onto the target body part for treatment, to determine, as a boundary region, a region in which a tissue structure has changed;
calculating variation in fluorescence intensity of the boundary region;
determining, based on the variation in the fluorescence intensity, whether or not additional application of the treatment light is to be performed;
applying the treatment light onto a region in which the additional application is required; and
calculating variation in fluorescence intensity of the boundary region after the additional application.

As explained above, a phototherapy device, a phototherapy method, and a computer-readable recording medium according to the disclosure are useful in appropriately applying a light onto the treatment region.

According to the disclosure, it becomes possible to appropriately apply light onto the treatment region.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A phototherapy device comprising:
a treatment light emitter configured to emit treatment light for causing a reaction of a drug;
a first imager configured to obtain a tissue structure image which is formed using narrow band light applied onto an application position of the treatment light;
a second imager configured to obtain a fluorescence image which is formed using excitation light applied onto the application position of the treatment light;
a boundary region calculator configured to refer to the tissue structure image to determine a boundary region in which a tissue structure has changed;
a fluorescence intensity variation calculator configured to calculate magnitude of variation in fluorescence intensity of the boundary region; and
a display image generator configured to generate a display image to be used for displaying the magnitude of variation of the fluorescence intensity.

2. The phototherapy device according to claim 1, wherein the boundary region calculator is configured to
detect a time variation in the tissue structure image, and
based on the time variation, determine, as the boundary region, a region of a body part in which the tissue structure has changed.

3. The phototherapy device according to claim 2, wherein the boundary region calculator is configured to
compare a value of the tissue structure image with a preset threshold value to determine, as the boundary region, the region of the body part in which the tissue structure has changed.

4. The phototherapy device according to claim 1, wherein the boundary region calculator is configured to
use a feature that has been calculated by a machine learning to determine, as the boundary region, a region of a body part in which the tissue structure has changed.

5. The phototherapy device according to claim 1, wherein the first imager is configured to obtain the tissue structure image which is formed using the narrow band light having a wavelength band equal to or greater than 380 nm and equal to or smaller than 440 nm.

6. The phototherapy device according to claim 1, further comprising a fluorescence intensity calculator configured to use a light intensity of returning light of narrow band light having a wavelength band equal to or greater than 440 nm and equal to or smaller than 490 nm to standardize the fluorescence intensity calculated by the fluorescence intensity variation calculator.

7. The phototherapy device according to claim 1, wherein the first imager is configured to obtain the tissue structure image which is formed using the narrow band light having a wavelength band equal to or greater than 490 nm and equal to or smaller than 590 nm.

8. The phototherapy device according to claim 1, wherein the first imager is configured to obtain the tissue structure image which is formed using the narrow band light having a wavelength band equal to or greater than 590 nm and equal to or smaller than 620 nm.

9. The phototherapy device according to claim 1, wherein the first imager is configured to obtain the tissue structure image which is formed using the narrow band light having a wavelength band equal to or greater than 620 nm and equal to or smaller than 780 nm.

10. The phototherapy device according to claim 1, further comprising a controller configured to control an application of the treatment light onto a target region for the application of the treatment light, while using cumulative value of light application intensity and light application period as a setting amount of applied light.

11. A phototherapy method implemented for applying treatment light, which causes a reaction of a drug, onto a treatment area to confirm an effect of treatment, the phototherapy method comprising:
obtaining a tissue structure image which is formed using narrow band light applied onto an application position of treatment light;
obtaining a fluorescence image which is formed using excitation light applied onto the application position of the treatment light;
referring to the tissue structure image to determine a boundary region in which a tissue structure has changed;
calculating magnitude of variation in fluorescence intensity of the boundary region; and
generating a display image to be used for displaying the magnitude of variation of the fluorescence intensity.

12. A non-transitory computer-readable recording medium that stores a computer program to be executed by a phototherapy device applying treatment light, which causes a reaction of a drug, onto a treatment area to generate information to be used in confirming an effect of treatment, the program causing the phototherapy device to execute:
obtaining a tissue structure image which is formed using narrow band light applied onto an application position of treatment light;
obtaining a fluorescence image which is formed using excitation light applied onto the application position of the treatment light;
referring to the tissue structure image to determine a boundary region in which a tissue structure has changed;
calculating magnitude of variation in fluorescence intensity of the boundary region; and
generating a display image to be used for displaying the magnitude of variation of the fluorescence intensity.
